(19) United States
(12) Patent Application Publication
(10) Pub. No.: US 2009/0061494 A1
(43) Pub. Date: Mar. 5, 2009

(54) Enzymatic Conversion of Epoxides to Diols

(76) Inventor: Kai-Uwe Schoning, Oberwil (CH)

Correspondence Address: JoAnn Villamizar;Ciba Corporation/Patent Department, 540 White Plains Road, P.O. Box 2005, Tarrytown, NY, 10591, US

(21) Appl. No.: 11/887,982

(22) PCT Filed: Apr. 3, 2006
(86) PCT No.: PCT/EP2006/061260

§ 371 Date: Oct. 5, 2007

(30) Foreign Application Priority Data
Apr. 11, 2005 (EP) 05102822.3

(51) Int. Cl. C12P 7/18; C12P 7/62

(52) U.S. Cl. 435/158; 435/135

(57) ABSTRACT

Diols of the formula I: or corresponding polymers wherein R₁ has been integrated into a polymeric backbone, wherein n ranges from (1) to (4); X is a divalent linking group selected from the group consisting of ether, ester, amino and amido groups; R₁ is an n-valent hydrocarbon residue containing at least one reactive group selected from the group consisting of carbon-carbon double or triple bonds and silyl groups; R₂, R₃ and R₄ independently are hydrogen or C₁-C₄alkyl; are conveniently prepared by treatment of an epoxide of the formula II wherein all residues and the index n are as defined for formula I, or a corresponding polymer, with an epoxide hydrolase, e.g. from lyophilized cells of Aspergillus niger.

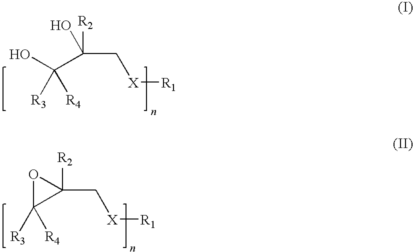

Present invention relates to a process for the preparation of reactive diols by enzymatic cleavage of a corresponding epoxide, and to the preparation of corresponding polymers.

The preparation of substituted glycols (substituted vicinal diols) from epoxides by classic acid or base catalyzed hydrolysis leads, especially with functional substituents, to undesired side products and low yields. Some specific epoxides have since been converted into the corresponding diols by cleavage with certain enzymes (e.g. Archelas et al., Topics in Current Chemistry 200, 159-191 (1999); EP-A-1291436; JP-A-11-032771).

An important class of epoxides or diols comprises compounds containing a further reactive functionality such as a carbon-carbon double bond or a silyl group separated from the epoxide by a heteroatom spacer. Examples for such difunctional compounds are those obtainable by reaction of a suitable silyl or ethylenically unsaturated precursor with epichlorohydrin. Compounds of this class may be converted into polyhydroxy functional polymers basically in 2 different ways, either

a) by converting the epoxide into the diol followed by incorporation of the diol into a polymeric structure retaining the hydroxy functionalities, or, vice versa, by
b) polymerization or copolymerization of the epoxide, followed by conversion of the polymeric epoxide into the polyhydroxy functional polymer.

It has now been found, that certain epoxides bonded by a heteroatom spacer to a polymerizable reactive group, or corresponding polymers or copolymers, may conveniently be converted into the corresponding diol or polyol in good yields.

Thus, present invention pertains to a process for the preparation of a monomeric compound of the formula I or a polymeric compound of the formula I′

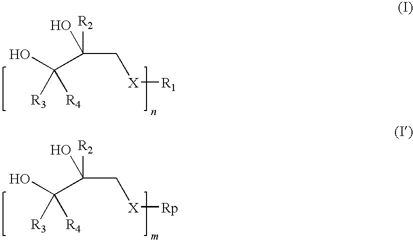

wherein
n ranges from 1 to 4;
m is the number of structural units derived from formula I where n is 1 in the compound of the formula I′, such as a number from the range 5 to about 10⁶;
X is a divalent linking group selected from the group consisting of ether, ester, amino and amido groups;
R₁ is an n-valent hydrocarbon residue containing at least one reactive group selected from the group consisting of carbon-carbon double bonds, carbon-carbon triple bonds and silyl groups;
R₂, R₃ and R₄ independently are hydrogen or C₁-C₄alkyl;
Rp is a polymeric backbone derived from the polymerization of compounds of the formula I where n is 1 and R₁ contains either a carbon-carbon double or triple bond or a silyl group, or by the copolymerization of such compounds of the formula I and one or more further suitable monomers;

which process comprises

a) treatment of a monomeric epoxide of the formula II

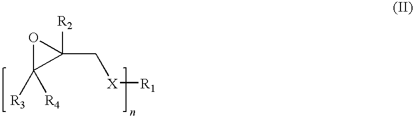

wherein all residues and the index n are as defined for formula I,
with an epoxide hydrolase, to obtain a compound of the formula I; or

b) treatment of a polymeric epoxide of the formula II′

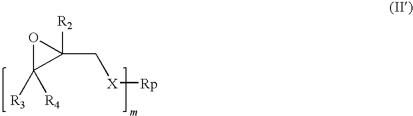

wherein all residues and the index m are as defined for formula I′,
with an epoxide hydrolase, to obtain a compound of the formula I′; or

c) treatment of a monomeric epoxide of the formula II wherein n is 1 with an epoxide hydrolase as described in variant a), and conversion of the compound of the formula I thus obtained by polymerization, or copolymerization with one or more further suitable monomers, into the compound of the formula I′,

provided that, in case that X stands for an ether group, the epoxide hydrolase has been isolated from a microorganism Aspergillus sp., especially Aspergillus niger such as the recombinant Fluka 71832.

Polyhydroxy functional polymers thus obtainable, e.g. hydrophilic or water soluble ones or those having a good capability of absorbing water and/or surface wetting, are useful in a wide range of end applications, inter alia including cosmetic preparations (such as described in JP-A-2001-172134, JP-A-10-310509, JP-A-10-310508, JP-A-10-310507, JP-A-11-071226); as a component for reprographic materials (e.g. paper, such as aqueous ink image receiving layers therein, U.S. Pat. No. 5,709,976; photographic or thermographic material, JP-A-62-042153; or inks, U.S. Pat. No. 5,268,027); water treatment, such as water softening or scale inhibition (EP-A-1158009); as hydrogels for cosmetic or household applications; for medical applications (such as medical adhesives, WO03025077, WO9740770, WO9702328, WO9703039; intraocular or contact lenses, WO01089423, WO9957582, JP-A-08-245737; therapeutic or diagnostic devices such as catheters, U.S. Pat. No. 5,603,991); organic-inorganic hybrid composites (U.S. Pat. No. 6,005,028); polymer films having high flexibility and blocking resistance (JP-A-09-208626).

Monomeric diols as of present formula I, besides their use as intermediates for the preparation of said polymers, themselves are useful inter alia as lubricants and/or antiwear additives.

Polyhydroxy functional polymers or, especially, oligomers (e.g. containing 2-10 monomer units corresponding to formula I′ wherein m is 2-10) or diols of formula (I) further are useful as metal chelating agents; the corresponding metal complexes (e.g. of Cu. Mo, Zn, Fe etc.) may find utility inter alia as micronutrients for agricultural purposes.

Examples for R₁ as an n-valent hydrocarbon residue containing at least one reactive group selected from the group consisting of carbon-carbon double or triple bonds and silyl groups include C₁-C₂₂hydrocarbons substituted by Si(OR₅)(OR₆)(OR₇), where R₅, R₆ and R₇ independently are H, C₁-C₈alkyl, cyclohexyl. Also included are C₃-C₂₂hydrocarbons containing an ethylenically unsaturated double bond or a carbon-carbon triple bond.

Thus, R₁ preferably is, in case that n is 1, C₁-C₂₂alkyl, C₆-C₁₂aryl, C₇-C₁₂arylalkyl, C₄-C₁₂cycloalkyl, C₅-C₁₂cycloalkylalkyl, C₆-C₁₂bicycloalkyl, C₇-C₁₂bicycloalkylalkyl, each of which is substituted; or R₁ is C₂-C₂₂alkenyl; C₂-C₈alkenyl-phenyl; C₂-C₂₂alkinyl; C₄-C₁₂cycloalkenyl, C₅-C₁₂cycloalkenylalkyl, C₆-C₁₂bicycloalkenyl, C₇-C₁₂bicycloalkenylalkyl, each of which is unsubstituted or substituted; and where substituents are selected from alkyl, alkoxy, alkanoyloxy, alkanoylamido, alkenoylamido, alkenyloxy, alkenyl, the substituents together containing 1 to 12 carbon atoms in total, or from —Si(OR₅)(OR₆)(OR₇), where R₅, R₆ and R₇ independently are H, C₁-C₈alkyl, cyclohexyl; with the proviso that R₁ contains at least one polymerizable double or triple bond or silyl group.

Preferred di-, tri or tetravalent residues R₁ corresponding to n as 2, 3 or 4 are derived from the above monovalent residues by abstraction of the appropriate number of hydrogen atoms. Examples are:

substituted C₁-C₂₂alkylene, C₆-C₁₂arylene, C₇-C₁₂arylalkylene, C₄-C₁₂cycloalkylene, C₅-C₁₂cycloalkylalkylene; or unsubstituted C₂-C₂₂alkenylene, C₂-C₈alkenyl-phenylene, C₂-C₂₂alkinylene, C₄-C₁₂cycloalkenylene (n=2);
substituted C₁-C₂₂alkan-triyl, trivalent C₆-C₁₂aryl, trivalent C₇-C₁₂arylalkyl, trivalent C₄-C₁₂cycloalkyl (n=3);
substituted C₁-C₂₂alkan-tetryl, tetravalent C₆-C₁₂aryl, tetravalent C₇-C₁₂arylalkyl, tetravalent C₄-C₁₂cycloalkyl, such as butane-1,2,3,4-tetryl (n=4).

In case that any residues are substituted, the substituents are mainly selected from alkyl, alkoxy, alkanoyloxy, alkanoylamido, alkenoylamido, alkenyloxy and/or alkenyl, and the substituents on one moiety usually contain 1 to 12 carbon atoms in total.

For example, monovalent R₁ (n=1) embraces C₁-C₂₂alkyl; C₂-C₂₂alkenyl; C₂-C₈alkenyl-phenyl; C₂-C₂₂alkinyl; C₄-C₁₂cycloalkyl, which is unsubstituted or substituted; C₄-C₁₂cycloalkenyl, which is unsubstituted or substituted; C₆-C₁₂aryl, which is unsubstituted or substituted; C₇-C₁₂arylalkyl, which is unsubstituted or substituted in the aromatic part; C₅-C₁₂cycloalkylalkyl, which is unsubstituted or substituted; C₅-C₁₂cycloalkenylalkyl, which is unsubstituted or substituted; C₆-C₁₂bicycloalkyl, which is unsubstituted or substituted; C₆-C₁₂bicycloalkenyl, which is unsubstituted or substituted; C₇-C₁₂bicycloalkylalkyl, which is unsubstituted or substituted; C₇-C₁₂bicycloalkenylalkyl, which is unsubstituted or substituted; and where each of these residues may be substituted by Si(OR₅)(OR₆)(OR₇), where R₅, R₆ and R₇ independently are H, C₁-C₈alkyl, cyclohexyl; with the proviso that R₁ contains at least one polymerizable double or triple bond or silyl group.

In compounds of the formula I or II of particular interest, n is 1 or 2; more preferably 1.

X preferably is COO or CONH with its carbon atom bonding to R₁, or is O.

In those compounds, the unit X—R₁ in case that n is 1 usually stands for C₃-C₈alkenyloxy; C₃-C₈alkenoyloxy; C₃-C₈alkenoylamido; cyclohexenyloxy; cyclohexenoyloxy; cyclohexenoylamido; or C₁-C₁₂alkoxy substituted by Si(OR₅)(OR₆)(OR₇) and optionally interrupted in a carbon-carbon, carbon-oxygen or carbon-silicon bond by cyclohexylene or phenylene, where R₅, R₆ and R₇ independently are H, C₁-C₈alkyl, cyclohexyl; and the unit X—R₁—X in case that n is 2 is O—C₂-C₄alkenylene-O, OOC—C₂-C₄alkenylene-COO, NHOC—C₂-C₄alkenylene-CONH, OOC-cyclohexylene-COO.

In most preferred compounds, each of R₂, R₃ and R₄ is hydrogen, or one of R₃ and R₄ is C₁-C₄alkyl while the others are hydrogen.

In important classes of compounds of the formula I or II, n is 1 and R₁ conforms to the formula V′

R₈—CH═C(R₅)—(R₇)_{q}— (V′)

wherein
R₅ and R₈ independently are H or methyl;
q is 1 or especially 0; and
R₇, if present, is C₁-C₄alkylene, phenylene or cyclohexylene; especially C₁-C₄alkylene.

Especially preferred units R₁—X— conform to the formulae

CH₂═C(R₅)—R₇—O— (VI′)

R₈—CH═C(R₅)—COO— (VII)

R₈—CH═C(R₅)—CONH— (VIII)

wherein R₅, R₇ and R₈ are as defined for formula V′.

Of special industrial importance is the conversion of glycidyl acrylate or glycidyl methacrylate according to the process of the invention.

Any alkyl, such as R₁ as C₁-C₂₂alkyl, within the definitions given embraces straight-chain or branched alkyl radicals, such as methyl, ethyl, n-propyl, isopropyl, n-butyl, sec-butyl, tert-butyl, amyl, isoamyl or tert-amyl, heptyl, octyl, isooctyl, nonyl, decyl, undecyl, dodecyl, tetradecyl, pentadecyl, hexadecyl, heptadecyl, octadecyl or eicosyl.

Alkoxy stands for alkyl linked over an oxygen atom as spacer: —O-alkyl.

C₂-C₂₂alkenyl includes, within the scope of the definitions given, inter alia vinyl, allyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl and n-octadec-4-enyl. Correspondingly, C₂-C₂₂alkinyl includes, within the scope of the definitions given, inter alia ethinyl, propinyl, butinyl, pentinyl, hexinyl, octinyl, etc.

Alkenyloxy stands for alkenyl linked over an oxygen atom as spacer: —O-alkenyl; it often embraces C₃-C₁₂alkenyloxy.

C₄-C₁₂cycloalkyl, which is unsubstituted or substituted, often embraces C₅-C₁₂cycloalkyl including cyclopentyl, cyclohexyl, cycloheptyl, cyclooctyl, cyclononyl, cyclodecyl, cycloundecyl and cyclodocecyl, and alkylated, especially methylated, variants of cyclopentyl, cyclohexyl, cycloheptyl, cyclooctyl. Cyclopentyl, cyclohexyl, cyclooctyl and cyclododecyl are preferred.

Cycloalkenyl mainly embraces C₅-C₁₂cycloalkenyl including cyclopentenyl, cyclohexenyl, cycloheptenyl, cyclooctenyl, cyclononenyl, cyclodecenyl, cycloundecenyl and cyclodocecenyl. Cyclohexenyl is preferred.

C₆-C₁₂aryl preferably is phenyl or naphthyl, especially phenyl; substituted variants include C₇-C₁₂alkylphenyl or alkenylphenyl such as styryl, C₁-C₄alkylphenyl.

C₃-C₈alkenoyloxy and C₃-C₈alkenoylamido include, for example, acryloyloxy, methacryloyloxy, acryloylamido, methacryloylamido, or corresponding alkylated variants up to the given number of carbon atoms.

Preferred divalent residues R₁ corresponding to n as 2 include:

1,2-vinylene, alkenylidenes such as 1,1-vinylene.

Preferably, R₁ contains only 1 polymerizable group of the specified classes.

The process of the invention preferentially leads to products of the formula I in racemic form. If desired, however, the instant diols (i.e. 2,3-dihydroxypropyl derivatives) may be obtained in enantiomerically enriched or even pure form, e.g. of either compound I(R) or I(S)

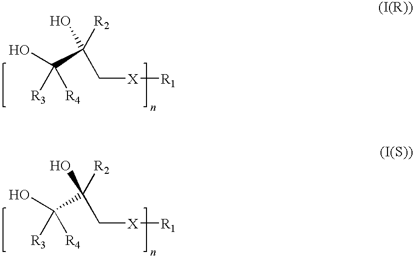

by either starting from enantiomerically enriched or pure starting materials or by careful selection of an appropriate enzyme or microorganism with preferention to formation of the desired (R) or (S) form. Alternatively, a subsequent racemate cleavage according to methods known in the art, such as chemical derivatization to diastereomers and subsequent separation, use of chemical chiral oxidation catalysts or oxidoreductases such as alcohol hydrogenases, or by performing chromatographical separation on chiral phases, or by a combination of these methods.

Enzymatic hydrolytic reactions are usually carried out in the presence of water, preferably in a solution, e.g. aqueous solution, especially buffer solution adapted to the working pH range of the selected microorganism or enzyme. In most cases, the pH ranges from about 4 to about 9, especially preferred is a neutral pH near pH 7, e.g. pH 6-pH 8. Aqueous solutions can be pure water or (preferably) buffered water solutions, or may be mixtures of water or water buffer with an organic solvent; generally suitable are all inert organic solvents, especially those miscible or partly miscible with water, e.g. those solvents showing miscibility with at least 1% by weight of water in the temperature range 10-50° C. The organic solvent usually is of lower polarity than water; examples are slightly polar hydrocarbons such as toluene, alcohols, ethers etc. as well as solvent mixtures. The reaction can be carried out in a homogeneous system or in multi phase systems, e.g. using 2 phases of solvent and/or a carrier-bound enzyme.

As silyl groups are sensitive to hydrolysis, compounds of the formula I, wherein R₁ contains a silyl group, are preferably prepared using mixtures of water and an organic solvent, e.g. containing 1-50% by weight, especially 1 to 20% by weight of water. More preferably, a two phase solvent system is used where the enzyme is present in the aqueous phase and the starting material and the product stay in the organic phase. Organic solvents preferentially used for phase transfer reactions include alkanes such as pentan and hexane, halogenated alkanes such as methylenchloride, alkanoles such as octonal, alkanones, aromatic solvents such as toluene, and ethers. More preferably, octanol and/or ether such as diisopropyl ether are used as organic phase. In some cases, it may be beneficial to add a further catalyst to the reaction system such as tetraalkyl ammonium salts, polyethylene glycols, crown ethers.

The present products of the formula I, especially those wherein n is 1, are useful for the preparation of specific polymers, which may be obtained by homopolymerization of the present products or, preferably, hetero(co)polymerization in combination with other suitable entities, for example ethylenically unsaturated monomers or oligomers such as (meth)acrylics; compounds of the formula I may also be used for modification/grafting on suitable polymers.

(Co)polymerizations are conveniently carried out following methods known in the art, e.g. addition (co)polymerization of compounds of formula I or II containing a carbon-carbon double bond under radical conditions, or by anionic or cationic (co)polymerization of said monomers, or by condensation (co)polymerizations of silyl monomers of formula I or II, optionally with further silyl monomers. Compounds of formula I or II containing a triple bond may be polymerized using radical initiators such as peroxides, (water soluble) rhodium complexes such as [Rh(norbornadiene)Cl]₂ in the presence of a base, or by cycloaddition reaction with e.g. pentadiene to yield the corresponding Diels-Alder product and subsequent ROM polymerization; see also (co)polymerizations of compounds containing a triple bond (denoted as non-volatile acetylenes) described in U.S. Pat. No. 6,093,779 or art cited therein. Preferred is radical polymerization of ethylenically unsaturated compounds.

Conversion of a monomeric epoxide of the formula II

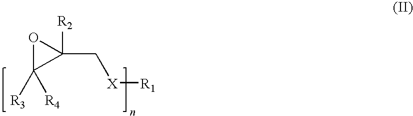

wherein all residues are as defined for formula I and the index n is 1, into the polymeric epoxide of the formula II′

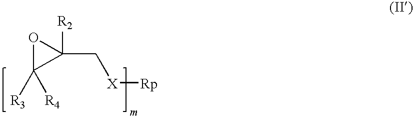

may be effected by polymerization or copolymerization with one or more further suitable monomers according to methods known in the art; the polymeric epoxide is then treated according to the invention with an epoxide hydrolase, to obtain a compound of the formula I′.

Suitable comonomers are those reactive with the polymerizable functional group of R₁, which preferably is a polymerizable carbon-carbon double bond. Compounds of the formula I containing a polymerizable carbon-carbon double bond may generally be converted into polyhydroxy functional polymers e.g. of the following classes, while monomers listed below may serve as comonomers for the preparation of copolymers as described above:

1. Polymers of monoolefins and diolefins, for example polypropylene, polyisobutylene, polybut-1-ene, poly-4-methylpent-1-ene, polyvinylcyclohexane, polyisoprene or polybutadiene, as well as polymers of cycloolefins, for instance of cyclopentene or norbornene, polyethylene (which optionally can be crosslinked), for example high density polyethylene (HDPE), high density and high molecular weight polyethylene (HDPE-HMW), high density and ultrahigh molecular weight polyethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, preferably polyethylene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, VIb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).

2. Mixtures of the polymers mentioned under 1), for example mixtures of polypropylene with polyisobutylene, polypropylene with polyethylene (for example PP/HDPE, PP/LDPE) and mixtures of different types of polyethylene (for example LDPE/HDPE).

3. Copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, ethylene/vinylcyclohexane copolymers, ethylene/cycloolefin copolymers (e.g. ethylene/norbornene like COC), ethylene/1-olefins copolymers, where the 1-olefin is generated in-situ; propylene/butadiene copolymers, isobutylene/isoprene copolymers, ethylene/vinylcyclohexene copolymers, ethylene/alkyl acrylate copolymers, ethylene/alkyl methacrylate copolymers, ethylene/vinyl acetate copolymers or ethylene/acrylic acid copolymers and their salts (ionomers) as well as terpolymers of ethylene with propylene and a diene such as hexadiene, dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EAA), LLDPE/EVA, LLDPE/EAA and alternating or random polyalkylene/carbon monoxide copolymers and mixtures thereof with other polymers, for example polyamides.

4. Hydrocarbon resins (for example C₅-C₉) including hydrogenated modifications thereof (e.g. tackifiers) and mixtures of polyalkylenes and starch.

Homopolymers and copolymers from 1.)-4.) may have any stereostructure including syndiotactic, isotactic, hemi-isotactic or atactic; where atactic polymers are preferred. Stereoblock polymers are also included.

5. Polystyrene, poly(p-methylstyrene), poly(α-methylstyrene).

6. Aromatic homopolymers and copolymers derived from vinyl aromatic monomers including styrene, α-methylstyrene, all isomers of vinyl toluene, especially p-vinyltoluene, all isomers of ethyl styrene, propyl styrene, vinyl biphenyl, vinyl naphthalene, and vinyl anthracene, and mixtures thereof. Homopolymers and copolymers may have any stereostructure including syndiotactic, isotactic, hemi-isotactic or atactic; where atactic polymers are preferred. Stereoblock polymers are also included.

6a. Copolymers including aforementioned vinyl aromatic monomers and comonomers selected from ethylene, propylene, dienes, nitriles, acids, maleic anhydrides, maleimides, vinyl acetate and vinyl chloride or acrylic derivatives and mixtures thereof, for example styrene/butadiene, styrene/acrylonitrile, styrene/ethylene (interpolymers), styrene/alkyl methacrylate, styrene/butadiene/alkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethylene/propylene/diene terpolymer; and block copolymers of styrene such as styrene/butadiene/styrene, styrene/isoprene/styrene, styrene/ethylene/butylene/styrene or styrene/ethylene/propylene/styrene.

6b. Hydrogenated aromatic polymers derived from hydrogenation of polymers mentioned under 6.), especially including polycyclohexylethylene (PCHE) prepared by hydrogenating atactic polystyrene, often referred to as polyvinylcyclohexane (PVCH).

6c. Hydrogenated aromatic polymers derived from hydrogenation of polymers mentioned under 6a.).

Homopolymers and copolymers may have any stereostructure including syndiotactic, isotactic, hemi-isotactic or atactic; where atactic polymers are preferred. Stereoblock polymers are also included.

7. Polymers derived from α,β-unsaturated acids and derivatives thereof such as polyacrylates and polymethacrylates; polymethyl methacrylates, polyacrylamides and polyacrylonitriles, impact-modified with butyl acrylate.

8. Copolymers of the monomers mentioned under 7) with each other or with other unsaturated monomers, for example acrylonitrile/butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.

9. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.

Most preferred comonomers are acrylics and vinyl derivatives such as compounds of the formula V

R₈—CH═C(R₅)—(R₇)_{q}—(X)ₚ—R₆ (V)

wherein
R₅ and R₈ independently are H or methyl;
X is a divalent linking group selected from the group consisting of ether, ester, amino and amido groups as defined for formula I;
p and q independently are 0 or 1;
R₆ is H, C₁-C₁₂alkyl, C₂-C₁₂hydroxyalkyl, phenyl;
R₇ is C₁-C₄alkylene, phenylene or cyclohexylene; especially C₁-C₄alkylene.

Especially preferred are comonomers conforming to the formulae

CH₂═C(R₅)—R₇—O—R₆ (VI)

R₈—CH═C(R₅)—COO—R₆ (VII)

R₈—CH═C(R₅)—CONH—R₆ (VIII)

wherein R₅, R₆, R₇ and R₈ are as defined for formula V.

Thus, typical polymer backbones Rp in compounds of formulae I′ or II′ are polyacrylics, vinyl polymer or mixed polyacryl-vinyl backbones derived from monomeric units of formulae I or II wherein R₁—X is acryloyloxy, methacryloyloxy, acryloylamido, methacryloylamido, C₃-C₆alkyleneoxy (homopolymers), or such backbones further containing comonomer units of acrylic acid, methacrylic acid, acrylamide, methacrylamide, acrylic esters, methacrylic esters, alkenes, alkenyl ethers (copolymers). Copolymers may be, for example, block or random type.

The following examples are for illustrative purposes only and are not to be construed to limit the instant invention in any manner whatsoever. Room temperature depicts a temperature in the range 20-25° C. Percentages are by weight unless otherwise indicated.

### EXAMPLE 1

0.5 g of allyl glycidyl ether (4.37 mmol) are dissolved in 100 ml phosphate buffer (20 mM, pH 7) and, under gentle agitation, 50 mg epoxide hydrolase recombinant from Aspergillus niger (Fluka 71832) are added. The reaction flask is placed on an orbital shaker and is shaken for 24 h at 50 rpm at 30° C. After this time, the starting material has completely vanished.

Comparison with commercially available monoallyl glycerol using thin layer chromatography, gas chromatography or high pressure liquid chromatography shows that only the diol has been obtained. The product is extracted with ethyl acetate, the organic phase dried with brine and over magnesium sulfate, and concentrated in vacuo to yield 0.55 g pure monoallyl glycerol (96%) as a clear colorless oil.

¹H-NMR (300 MHz, CDCl₃=7.25 ppm): 5.85 (1H), 5.22 (1H), 5.14 (1H), 3.96 (2H), 3.83 (1H), 3.58 (2H), 3.44 (2H)

13C-NMR (175 MHz, CDCl3=77.4 ppm): 134.5, 117.6, 72.6, 71.8, 71.1, 64.2.

The same product is obtained using lyophilized cells (1000 mg, respectively) of

a) Aspergillus niger,
b) Rhodococcus ruber (DSM 43338),
c) Myobacterium paraffinicum (NCIMB 10420),
d) Bacillus megaterium (DSM 32),
e) Rhodococcus erythropolis (DSM 9685),

### f) Corynebacterium sp. (DSM 20415),

g) L. piscicola (L. carnis; DSM 20722)
under otherwise same conditions in varying yields.

### EXAMPLES 2-7

Following the general method shown in example 1, the substrates shown in table 1 can be converted.

### EXAMPLE 8

0.5 g of (3-glycidoxypropyl)triethoxysilane (1.8 mmol) is dissolved in 50 ml 1-octanol and added to a solution of 500 mg epoxide hydrolase recombinant from Aspergillus niger (Fluka 71832) in 20 ml phosphate buffer (20 mM, pH 7). The reaction medium is stirred at 200 rpm by the aid of an external mixer at 18° C. for 24 h. After this time, the phases are allowed to separate and the organic phase is thoroughly dried over magnesium sulfate. The solvent is removed in vacuo to yield a mixture of 3-[3-(triethoxysilyl)propoxy]-1,2-propanediol (see table 1) and 3-glycidoxypropyl)triethoxysilane, which can be separated by distillation. Yield: 64%.

²⁹Si-NMR: 40.3, 40.5, 41.0, 41.5

TABLE 1
Examples 2-8

| Example No. | Substrate | Product | Yield |
|---|---|---|---|
| 2 | 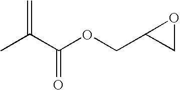 | 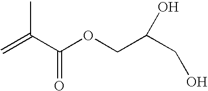 | 93% |
| 3 | 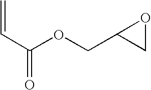 | 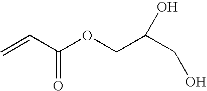 | 95% |
| 4 | 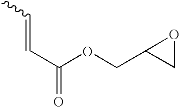 | 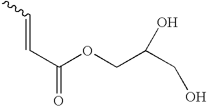 | 88% |
| 5 | 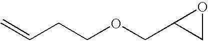 | 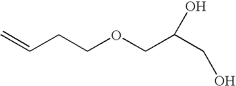 | 91% |
| 6 | 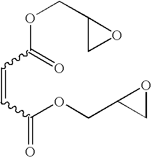 | 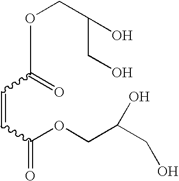 | 53% |
| 7 | 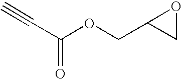 | 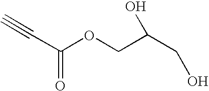 | 93% |
| 8 | 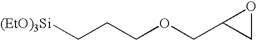 | 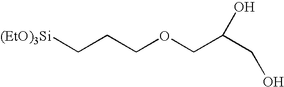 | 64% |

Epoxide hydrolases can be obtained from bacterial, yeast and fungal sources as well as from mammalian cells; whole cells may be used (e.g. lyophilized cells), or the enzyme in isolated form. Cell preparations or isolated/recombinant enzymes are widely known, many are commercially available.

Examples for microorganisms producing suitable epoxide hydrolases are genera:

### Actinomyces sp.

### Rhodococcus sp.

### Nocardia sp.

### Methylobacterium sp.

### Macoplana sp.

### Rhodotorula sp.

### Rhodosporidium sp.

### Aspergillus sp.

### Beauveria sp.

### Pseudomonas sp.

### Chryseomonas sp.

### Agrobacterium sp.

### Diplodia sp.

### Solanum sp.

### Mortierella sp.

### Trichosporon sp.

### Arthrobacter sp.

### Mycobacterium sp.

### Corynesporium sp.

### Helminthsporium sp.

### Bacillus sp.

### Variovorax sp.

### Lactobacillus sp.

Most preferred is the use of epoxy hydrolases of Aspergillus sp. such as Aspergillus niger, especially the recombinant species as in the above examples (Fluka 71832), either in isolated form or as whole cells.

### EXAMPLE 9

### a) Synthesis of a Copolymer of Glycidyl Acrylate and Acrylamide

In a 50 ml round bottomed flask, acrylamide (0.6 g, 8.3 mmoles) and 2,2′-azobis(2-methylbutyronitrile) (17 mg, 0.1 mmole) are dissolved in 8.5 ml tetrahydrofuran. To this solution is added 0.35 g (2.77 mmoles) of glycidyl acrylate. Argon is bubbled through the solution for 5 minutes, the flask is tightly closed and heated over night at 64° C. under thorough mixing. The solution is concentrated in vacuo and the precipitated polymer collected by filtration. The polymer is resuspended in a mixture of tetrahydrofuran and methanol (1:1), thoroughly extracted, collected by filtration, washed with pure methanol and methylenechloride and dried in vacuo at 35° C.

Measurement of an IR-spectrum reveals that glycidyl acrylate is incorporated in the polymer (IR bands at 3050, 1250, 910, and 800 cm⁻¹).

### b) Enzymatic Ring Opening in Copolymeric Epoxide

The copolymer (0.1 g) is suspended in 5 ml of phosphate buffer (pH7) and 0.5 ml of DMSO. The mixture is adjusted to 30° C. in an orbital shaker, and 50 mg of epoxide hydrolase recombinant from Aspergillus niger (Fluka 71832) are added. The reaction mixture is shaken for 7 days, during that time an additional 25 mg of enzyme are added. The polymer is separated by centrifugation and washed successively with 0.1 M sodium carbonate solution, deionized water, methanol. The polymer is dried at 35° C. in an vacuum oven. Analysis by IR spectroscopy indicates that epoxide band are not present anymore, instead new signals in the region of 3380 cm⁻¹ prove the existence of hydroxy groups.

### EXAMPLE 10

### Synthesis of a Copolymer of Acrylic Acid and Monoallyl Glycerol

To 30 ml of deionized water are added simultaneously at 100° C. over a period of 3.5 h: 93 g of a 20% solution of sodium acrylate, 20 ml of an 8% aqueous solution of monoallyl glycerol (product of example 1) and 14 ml of a 10% aqueous solution of ammonium persulfate. After addition of the solutions, the reaction mixture is kept at 100° C. for an additional 60 minutes to allow for the completion of the polymerization. A yellowish viscous solution is obtained. The polymer is isolated upon addition of hydrochloric acid (pH 2-3) and addition of organic solvents to cause precipitation.

1. A process for the preparation of a monomeric compound of the formula I or a polymeric compound of the formula I′ 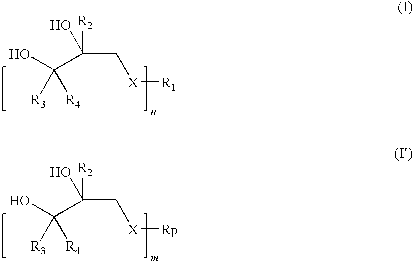
wherein
n ranges from 1 to 4;
m is the number of structural units of the formula I where n is 1 in the compound of the formula I′, such as a number from the range 5 to about 10⁶;
X is a divalent linking group selected from the group consisting of ether, ester, amino and amido groups;
R₁ is an n-valent hydrocarbon residue containing at least one reactive group selected from the group consisting of carbon-carbon double bonds, carbon-carbon triple bonds and silyl groups;
R₂, R₃ and R₄ independently are hydrogen or C₁-C₄alkyl;
Rp is a polymeric backbone derived from the polymerization of compounds of the formula I where n is 1 and R₁ contains either a carbon-carbon double or triple bond or a silyl group, or by the copolymerization of such compounds of the formula I and one or more further suitable monomers;
which process comprises
a) treatment of a monomeric epoxide of the formula II 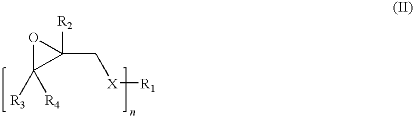
wherein all residues and the index n are as defined for formula I,
with an epoxide hydrolase, to obtain a compound of the formula I; or
b) treatment of a polymeric epoxide of the formula II′ 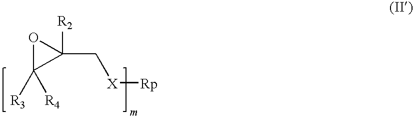
wherein all residues and the index m are as defined for formula I′,
with an epoxide hydrolase, to obtain a compound of the formula I′; or
c) treatment of a monomeric epoxide of the formula II wherein n is 1 with an epoxide hydrolase as described in variant a), and conversion of the compound of the formula I thus obtained by polymerization, or by copolymerization with one or more further suitable monomers, into the compound of the formula I′;
provided that, in case that X stands for an ether group, the epoxide hydrolase is isolated from a microorganism Aspergillus sp.

2. A process according to claim 1, wherein the hydrolase is isolated from the group consisting of bacteria, yeast, fungal sources, and mammalian cells.

3. A process according to claim 2, wherein the hydrolase is isolated from a microorganism selected from the group consisting of Actinomyces sp., Rhodococcus sp., Nocardia sp., Methylobacterium sp., Macoplana sp., Rhodotorula sp., Rhodosporidium sp., Aspergillus sp., Beauveria sp., Pseudomonas sp., Chryseomonas sp., Agrobacterium sp., Diplodia sp., Solanum sp., Mortierella sp., Trichosporon sp., Arthrobacter sp., Mycobacterium sp., Corynesporium sp., Helminthsporium sp., Bacillus sp., Variovorax sp., Lactobacillus sp., and Aspergillus niger.

4. A process according to claim 1, where in the compounds of formulae I and II R₁ embraces, in case that n is 1, C₁-C₂₂alkyl, C₆-C₁₂aryl, C₇-C₁₂arylalkyl, C₄-C₁₂cycloalkyl, C₅-C₁₂cycloalkylalkyl, C₆-C₁₂bicycloalkyl, C₇-C₁₂bicycloalkylalkyl, each of which is substituted; or R₁ is C₂-C₂₂alkenyl; C₂-C₈alkenyl-phenyl; C₂-C₂₂alkinyl; C₄-C₁₂cycloalkenyl, C₅-C₁₂cycloalkenylalkyl, C₆-C₁₂bicycloalkenyl, C₇-C₁₂bicycloalkenylalkyl, each of which is unsubstituted or substituted; and where substituents are selected from alkyl, alkoxy, alkanoyloxy, alkanoylamido, alkenoylamido, alkenyloxy, alkenyl, the substituents together containing 1 to 12 carbon atoms in total, or from —Si(OR₅)(OR₆)(OR₇), where R₅, R₆ and R₇ independently are H, C₁-C₈alkyl, cyclohexyl; with the proviso that R₁ contains at least one polymerizable double or triple bond or silyl group; and where di-, tri or tetravalent residues R₁ corresponding to n as 2, 3 or 4 are derived from the above monovalent residues by abstraction of the appropriate number of hydrogen atoms.

5. A process according to claim 1, where in the compounds of formulae I and II n is 1 or 2 and
the unit X—R₁, in case that n is 1, is C₃-C₈alkenyloxy; C₃-C₈alkenoyloxy; C₃-C₈alkenoylamido; cyclohexenyloxy; cyclohexenoyloxy; cyclohexenoylamido; or C₁-C₁₂alkoxy substituted by Si(OR₅)(OR₆)(OR₇) and optionally interrupted in a carbon-carbon, carbon-oxygen or carbon-silicon bond by cyclohexylene or phenylene, where R₅, R₆ and R₇ independently are H, C₁-C₈alkyl, cyclohexyl; and
the unit X—R₁—X, in case that n is 2, is O—C₂-C₄alkenylene-O, OOC—C₂-C₄alkenylene-COO, NHOC—C₂-C₄alkenylene-CONH, or OOC-cyclohexylene-COO.

6. A process according to claim 5, where in the compounds of formulae I and II n is 1,
each of R₂, R₃ and R₄ is hydrogen, or one of R₃ and R₄ is C₁-C₄alkyl while the others are hydrogen; and
X—R₁ is of the formula VII or VIII
R₈—CH═C(R₅)—COO— (VII)
R₈—CH═C(R₅)—CONH— (VIII)
wherein R₅ and R₈ independently are H or methyl.

7. A process according to claim 6 for the preparation of glycerol monoacrylate or glycerol monomethacrylate.

8. A process according to claim 1, wherein the treatment with epoxide hydrolase is carried out in the presence of water, and optionally an organic solvent, in the temperature range 10-50° C.

9. A process according to claim 1, where in for the compounds of formulae I′ and II′ Rp is a homopolymer backbone derived from compounds of the formula I or II wherein R₁ contains a polymerizable carbon-carbon double bond, or a copolymer derived from compounds of the formula I or II, wherein R₁ contains a polymerizable carbon-carbon double bond, and one or more further monomers conforming to the formula V
R₈—CH═C(R₅)—(R₇)_{q}—(X)ₚ—R₆ (V)
wherein
R₅ and R₈ independently are H or methyl;
X is a divalent linking group selected from the group consisting of ether, ester, amino and amido groups as defined for formula I;
p and q independently are 0 or 1;
R₆ is H, C₁-C₁₂alkyl, C₂-C₁₂hydroxyalkyl, phenyl;
R₇ is C₁-C₄alkylene, phenylene or cyclohexylene.

10. A process according to claim 9 for the preparation of a polymeric compound of the formula I′ starting from a monomeric epoxide of the formula II, wherein n is 1 and R₁ conforms to the formula V′
R₈—CH═C(R₅)—(R₇)_{q}— (V′)
wherein
R₅ and R₈ independently are H or methyl;
q is 0 or 1; and
R₇ is C₁-C₄alkylene, phenylene or cyclohexylene;
by converting the monomer of formula II into a polymer of the formula II′ and subsequent enzymatic reaction by treatment of a Polymeric epoxide of the formula II′ 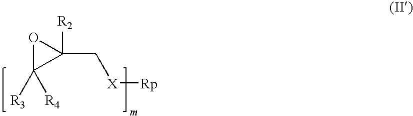
wherein all residues and the index m are as defined for formula I′,
with an epoxide hydrolase, to obtain a compound of the formula I′: or
by carrying out the enzymatic reaction and subsequent polymerization by treatment of a monomeric epoxide of the formula II wherein n is 1 with an epoxide hydrolase as described in variant a), and conversion of the compound of the formula I thus obtained by polymerization, or by copolymerization with one or more further suitable monomers, into the compound of the formula I′.

11. A process according to claim 1 for the preparation of a polymeric compound of the formula I′, wherein Rp is a polymeric backbone selected from the group consisting of polyacrylics, vinyl polymer and mixed polyacryl-vinyl backbones, which are homopolymeric backbones derived from monomeric units of formulae I or II wherein R₁—X is acryloyloxy, methacryloyloxy, acryloylamido, methacryloylamido, C₃-C₆alkyleneoxy, or copolymeric backbones further containing one or more comonomer units selected from the group consisting of acrylic acid, methacrylic acid, acrylamide, methacrylamide, acrylic esters, methacrylic esters, alkenes, and alkenyl ethers.

12. A method for the enzymatic conversion of an epoxy-functional polymer into a polyol wherein said method comprises contacting said epoxy-functional polymer with an effective catalyzing amount of a hydrolase from a microorganism selected from the group consisting of Actinomyces sp., Rhodococcus sp., Nocardia sp., Methylobacterium sp., Macoplana sp., Rhodotorula sp., Rhodosporidium sp., Aspergillus sp., Beauveria sp., Pseudomonas sp., Chryseomonas sp., Agrobacterium sp., Diplodia sp., Solanum sp., Mortierella sp., Trichosporon sp., Arthrobacter sp., Mycobacterium sp., Corynesporium sp., Helminthsporium sp., Bacillus sp., Variovorax sp., and Lactobacillus sp.

13. A process according to claim 3, wherein the hydrolase is isolated from the microorganism Aspergillus niger.

14. A process according to claim 8, wherein the treatment with epoxide hydrolase is carried out in the presence of an aqueous buffer solution at a pH between about 4 and about 9, and optionally an organic solvent, in the temperature range 10-50° C.

15. A process according to claim 9, where in for the compounds of formulae I′ and II′ Rp is a homopolymer backbone derived from the radical polymerization of compounds of the formula I or II wherein R₁ contains a polymerizable carbon-carbon double bond, or a copolymer derived from compounds of the formula I or II, wherein R₁ contains a polymerizable carbon-carbon double bond, and one or more further monomers conforming to the formula V
R₈—CH═C(R₅)—(R₇)_{q}—(X)ₚ—R₆ (V)
wherein
R₅ and R₈ independently are H or methyl;
X is a divalent linking group selected from the group consisting of ether, ester, amino and amido groups as defined for formula I;
p and q independently are 0 or 1;
R₆ is H, C₁-C₁₂alkyl, C₂-C₁₂hydroxyalkyl, phenyl;
R₇ is C₁-C₄alkylene.
